(19) United States
(12) Patent Application Publication
(10) Pub. No.: US 2009/0215883 A1
(43) Pub. Date: Aug. 27, 2009

(54) Pharmaceutical formulation comprising taxane, a solid composition of taxane, a process for preparing said solid composition of taxane, a solubilizing composition of said solid composition of taxane, and a kit for the injectable formulation of taxane

(75) Inventors: Antonio Osvaldo Bouzada, Parana (AR); Jose Lucio Nunez, Parana (AR); Jose Bernardo Iturraspe, Santa Fe (AR); Nora Adriana Moyano De Iturraspe, Santa Fe (AR)

Correspondence Address: RATNERPRESTIA, P.O. BOX 980, VALLEY FORGE, PA, 19482, US

(73) Assignee: ERIOCHEM S.A., Paranas, Entre Rios (AR)

(21) Appl. No.: 12/161,625

(22) PCT Filed: Jan. 18, 2007
(86) PCT No.: PCT/ES2007/070012

§ 371 Date: Oct. 20, 2008

(30) Foreign Application Priority Data
Jan. 20, 2006 (AR) P06100208

(51) Int. Cl. A61K 31/337; C07D 305/08

(52) U.S. Cl. 514/449; 549/510

(57) ABSTRACT

A pharmaceutical formulation of taxane intended to be administered to mammals, preferably humans, comprises two compositions combined prior to being administered, forming a transparent solution free from precipitates, in which the compositions comprise a solid composition of lyophilized taxane, free from tensoactives, oils, polymers, solubility enhancers, preservatives and excipients; and a solubilizing composition of the lyophilized taxane solid composition that comprises at least one tensoactive. This formulation is free from polysorbate 80 and polyoxyethylated castor oil. A procedure is provided for the preparation of the solid composition by means of the lyophilization of taxane in a lyophilizing organic solvent. A kit for the injectable formulation of taxane comprises a prefilled syringe. Also a pharmaceutical taxane solution for perfusion, free from organic solvent, is provided.

### FIELD OF THE INVENTION

This invention belongs to the field of the formulations of pharmaceutical drugs which are poorly soluble in water. Particularly, it refers to oncological drug formulations, in which said drugs belong to the taxane group. More specifically, the invention is directed to formulations intended for parenteral infusion processes typical of oncological chemotherapy with docetaxel and paclitaxel.

### STATE OF THE ART

The pharmacological formulations of drugs which are poorly soluble in aqueous media have been extensively studied over the last decades. Innumerable strategies have been developed in order to inject these drugs into mammals with the aim of improving their pharmacotechnical properties and ameliorate their side effects.

Concerning formulations which are suitable for the preparation of solutions for parenteral infusion over a long period, and especially for oncological chemotherapy treatments, technical problems arise, namely as how to maintain these drugs in the aqueous solution of the parenteral infusion for periods of at least 4 hours for conventional infusion protocols and at least for 72 hours for administration by means of a continuous infusion pump. Another problem described is the in situ gelification which occurs at the time of injecting the drug solution in the presence of high tensoactive concentrations, in the container of the parenteral infusion. This phenomenon is described as irreversible, for the tensoactive gelifies and does not solubilize in the parenteral solution.

Of a particular interest for this invention are the compounds of the taxane family, products extracted from the leaves and bark of a tree commonly known as European yew (Taxus Baccata and other species of the Taxus family) as paclitaxel, as well as semi-synthetic products obtained from baccatin III or from 10-deacetylbaccatin III which are also extracted from yew, like docetaxel. Those taxanes obtained from biotechnological processes are also of interest for the present invention.

The medical uses of taxanes are varied; their anti-tumoral effects can be mentioned among others. Some examples of cancer which can be treated with docetaxel are: locally advanced or metastatic cancer, breast cancer, non-small cell lung cancer, hormone-refractory prostate cancer and ovarian cancer, and gastric cancer.

U.S. Pat. No. 4,814,470 to Colin, Michel et al., from the company Rhône-Poulenc Santé(Courbevoie, FR), refers to a taxane pharmaceutical composition from which 10-deacetylbaccatin III derivatives are obtained, specially docetaxel. This document describes a synthesis that ends with a crystallization and a formulation comprising the dissolution of the crystals obtained in a mixture of equal parts of non-ionic tensoactives and alcohol. This formulation gelifies when injected into the parenteral infusion bag.

A series of patents from the company Rhône-Poulenc Santé(Courbevoie, FR) protects the current formulation of docetaxel (U.S. Pat. No. 5,438,072, U.S. Pat. No. 5,698,582, U.S. Pat. No. 5,714,512 and U.S. Pat. No. 5,750,561). This technology solves the in situ precipitation and gelification problems by a formulation having two solutions, one of taxane in polysorbate 80 and alcohol (which can be present in very low concentrations) and the other of water and ethanol. These documents describe a preparation process which consists of mixing both solutions without intense stirring, to generate a solution of Taxotere 10 mg/ml (stable between 2 and 25° C. for 4 hours), which solves the problem of gelification in situ by diluting such solution in an aqueous 5% dextrose solution or normal saline solution of sodium chloride at 0.9% to obtain the infusion solution at a concentration of 0.3 to 0.74 mg/ml (stable for 8 hours, between 2 and 25° C.).

This liquid formulation of docetaxel in polysorbate 80 (in the presence or absence of alcohol) poses the problem of stability over time; being at room temperature between 2 and 25° C. recommended for its conservation. Besides, the presence of polysorbate 80 causes known adverse side effects due to the incorporation of said tensoactive in high concentrations—which are necessary to maintain the drug in solution—into the blood stream. The same happens with the formulation of paclitaxel available on the market, which requires high concentrations of Cremophor. The adverse side effects caused by these tensoactives present in taxane formulations currently available, particularly docetaxel and paclitaxel, require a pre-treatment with steroids or antihistamines before oncological chemotherapy as described in the literature, as follows: ‘ten Tije A J, Verweij J, Loos W J, Sparreboom A. Pharmacological Effects of Formulation Vehicles: Implications for Cancer Chemotherapy. Clin Pharmacokinet 2003; 42: 665-685’; ‘Rowinsky E K, Eisenhauer E A, Chaudhry V et al. Clinical toxicities encountered with paclitaxel (Taxol). Semin Oncol 1993, 20: 1-15’; ‘Bernstein B. Docetaxel as an Alternative to Paclitaxel after Acute Hypersensitivity Reactions. Ann Pharmacother 2000; 34:1332-1335’; ‘Novel Formulations of Taxanes: A review. Old Wine in a New Bottle? K. L. Hennenfentl & R. Govindan Annals of Oncology 17: 735-749, 2006 doi: 10.1093/annonc/mjd 100 Published on line 19 December, 2005’.

Likewise, the formulation of docetaxel currently available on the market requires for its use a process which involves several steps and a certain risk for the doctors and nurses involved in its administration. These steps, aimed at ensuring the proper administration of this drug means: extracting the solvent from an ampoule and introducing it into a vial containing docetaxel solution, gently agitating to homogenize the solution, allowing a rest time for foam to disappear, extracting the mix and injecting it into the perfusion bag or flask filled with saline solution or dextrose, homogenizing and finally inspecting it for possible precipitate formation before administering it to the patient (should precipitation appear, the solution must be disposed of with the resulting economical negative impact).

The whole process must be carried out aseptically. The risks of contamination are high and the operation requires trained personnel and considerable time. There is also a risk of contamination for health care providers in contact with the cytotoxic solution handled, due to the aerosolization of the drug.

Currently, there is a need to simplify such process of administration, to shorten the time for the preparation of the perfusion formulation and to decrease the risks implied.

The toxic effects of the tensoactives used in the formulation of docetaxel and paclitaxel, such as polyoxyethylated castor oil (Emulphor® or Cremophor®) or polysorbate 80 (Tween 80®) are known. The pharmacological and biological effects caused by these tensoactives have been described as acute hypersensitivity reactions, peripheral neuropathy, cumulative fluid retention syndrome, etc. A great number of patients cannot be treated due to the side effects of such tensoactives, eg. patients presenting hypersensitivity, patients with impaired renal function, elderly patients, patients suffering from a cardiopathy, etc. These tensoactives also affect the availability of the drugs which are solubilized and administered intravenously.

That is the reason why great efforts have been made in the scientific field in order to find formulations which prevent or decrease the use of said tensoactives. Among the strategies described in the state of the art, we can mention the development of albumin nanoparticles, polyglutamates, taxane analogs, prodrugs, emulsions, liposomes, etc.

For the purpose of reference, literature is provided to offer a detailed description of the latest development and clinical tests: ‘Novel formulations of taxanes: A Review. Old Wine in a New Bottle?’ K. L. Hennenfentl & R. Govidan 2 1^{st} Louis college Pharmacy. Ortho Biotech Clinical Affairs, LLC, St Louis Mo.; 2 Alvin J Siteman Cancer Center, Washington University School of Medicine, St. Louis, Mo., USA accepted 7 Nov., 2005. Other works contributing solutions to these problems are: Castro C A et al. Pharmacol Biochem Behav, 1995 April; 50(4):521-6; Sanzgiri U Y et al., Fundam Appl Toxicol, 1997 March; 36(1):54-61; ten Tije A J et al., Clin Pharmacokinet. 2003; 42(7):65-85: Constantine J W et al., Experientia. 1979 Mar. 15; 35(3):338-9: Van Zuylen L et al., Invest New Drugs 2001 May; 19(2):125-41: Bergh M et al., Contact Dermatitis. 1997 July; 37(1):9-18).

The WHO has estimated that the maximum daily dose of polysorbate 80 is 25 mg/kg of body weight (FAO WHO. Tech. Rep. Ser. Wld. Hlth. Org. 1974, No 539). Therefore, a man weighing 75 kg may be administered 1.875 g of polysorbate 80 per day; i.e., for a dosage form of 75 mg of Taxotere® per m² commonly used for lung or prostate cancer, a man who is 1.75 m tall and weighs 75 kg, has a body surface of 1.90 m² and is to be administered 143 mg of docetaxel along with approximately 3.6 g of polysorbate 80 (40 mg of docetaxel per ml of Tween 80®), i.e. almost 2-fold the maximum daily dosage form of polysorbate 80 recommended by the WHO.

On the other hand, lyophilized formulations of certain injectable drugs tend to be more advantageous than injectable liquid formulations, particularly in those cases in which the lyophilized solution is of a higher chemical and physical stability, i.e, it has a longer shelf-life and is more resistant to higher temperatures, as those in the warm climates of the regions belonging to Zone 4, according to the International Committee for Harmonization (ICH).

Particularly, the process of lyophilization of drugs which are poorly soluble in water, mainly taxanes, presents great difficulties because the standard techniques of lyophilization consist of freezing aqueous solutions and subjecting them to vacuum to achieve sublimation. Apart from water there are not many solvents which allow this procedure within acceptable pharmacotechnical conditions. In order to be lyophilized, a good formulation should not pose “puffing” problems, i.e, when frozen it should not generate a plastic solid which bubbles on sublimation. Furthermore, it should be a good heat conductor and the solvent should generate a lyophilized plug of suitable pharmacotechnical characteristics. Moreover, the material structure should meet the condition of having spaces to allow the diffusion of the gas produced by the sublimation of the solvent through the cake. Besides, the plug should be rigid enough to support its own structure, aided by an excipient if needed.

There have been many attempts in the state of the art in order to solve the problem of toxicity of non-ionic tensoactives, proposing taxane lyophilized formulations. For example, patent WO 99/24073 by Géczy, J (Thissen Laboratories S.A.) proposes to lyophilize docetaxel and paclitaxel starting from a hydroalcoholic solution of these drugs and cyclodextrins. This solution allows to obtain a lyophilizate containing taxane complexed to a lyophilized powder cyclodextrin (it uses 2hydroxypropyl β-cyclodextrin for docetaxel in a mass ratio of approximately 1:100 of active principle:cyclodextrin). This lyophilizate can be dissolved in an aqueous solution of up to 1 mg/ml, ready to perfuse. Although this eliminates the use of non-ionic tensoactives, this formulation incorporates into the blood stream a complex between taxane and cyclodextrin which not only increases stability as regards precipitation of an oversaturated aqueous solution of taxane, but which can also modify the significant pharmacodynamic properties of taxane. Therefore, a toxicological and clinical study is required to endorse the use of this new complex between taxane and cyclodextrin. Besides, it involves complex elaboration processes.

Other attempts to obtain taxane lyophilizates are shown in the state of the art, such as the application for patent US20030099674 by Chen, Andrew in which obtaining a lyophilized taxane from an oil/water emulsion using lecithin as a tensoactive and sucrose as an anti-adhesion agent is proposed. Bile salts are among the surfactants mentioned in the description. Taxane is dissolved in ethanol and water, and the solvents are then eliminated by lyophilization, generating a taxane liposome when it is reconstituted with water.

Furthermore, the application of patent US20030099675 by Jeong, Seo Young, proposes a liquid formulation having an organic solvent, an emulsifier and a monoglyceride. It also proposes forming an emulsion in water from a liquid formulation and lyophilizing it. The use of active drugs such as paclitaxel is mentioned in this document.

In these last two applications a taxane lyophilizate with a good solubility in water is obtained. However, said taxane lyophilizate poses similar problems to those mentioned before as regards the use of taxane emulsions which can modify their pharmacodynamics. Naturally, emulsions and microemulsions as liposomes generate autoimmune responses when administered endogenously and are attacked by macrophages, which causes an important part of the dosage to be unavailable for the desired action apart from generally requiring a pretreatment with steroids or antihistamines.

Other technological development as the one described in the application US20030187062 by Zenoni Mauricio, et al. from ACS DOBFAR S.A. proposes obtaining micro or nanoparticles by lyophilizing paclitaxel and albumin.

There is a vast literature describing taxane liposomes as in patent EP1332755, in which a lyophilizate of paclitaxel is obtained using a compound like lecithin or cholesterol in a solution of isopropanol or ethanol in order to subsequently obtain liposomes which are always lyophilized from aqueous solutions. One of the drawbacks of these developments is that they modify the pharmacodynamics of taxane, as they have a short useful life and require a cold chain for their preservation, apart from generating an immune response and causing the attack of macrophages which decreases the effect of the drug considerably.

There are many patents claiming polymer micellae in the literature, namely U.S. Pat. No. 5,543,158 and U.S. Pat. No. 6,322,805. In particular, U.S. Pat. No. 6,322,805 refers to obtaining biodegradable polymeric micellae capable of solubilizing hydrophobic drugs comprising amphiphilic block copolymers, which have a hydrophilic polyalkyl oxide and a biodegradable hydrophobic polymer selected from the group consisting of polylactic acid, polyglycolic, polylactic-co-glycolic acid, poly(epsilon-caprolactone) derivatives and the mixtures thereof. It describes how the hydrophobic drug is trapped in the micelle in the absence of a covalent bond. These micellae form a solution in water acting as solubilizing agent. This solution can be lyophilized, preserved and reconstituted with water or isotonic solution. This patent does not solve the problem of increasing the solubility of taxane as such, but it presents a complex process of synthesis of a specific copolymer to generate taxane micellae. In spite of having done promising tests regarding the characteristics of stability, components are added to the drug thus radically changing not only the bioavailability but also the kinetics of the original taxane. This patent does not describe the method to obtain a sterile solution by means of reconstitution.

The document U.S. Pat. No. 6,780,324 describes a process in which a solution of a biologically active hydrophobic agent is formed in combination with a dispersing agent and an organic solvent or even a mixture in which water can be included. This mixture can be lyophilized and redissolved to form a nanodispersion or a micelle solution. Drug is not lost during this process; it can be sterilized by filtration. A transparent solution is obtained by reconstitution but the lyophilizate has other components apart from the drug, thus posing risks not only for its chemical stability but also for its bioavailability. On the other hand, the procedure proposed requires operations such as sonication, intense stirring and heating to obtain a solution to be lyophilized which can either destroy micellae or cause taxane degradation.

U.S. Pat. No. 6,610,317 B2 by Julie Straub et al. describes a porous matrix of paclitaxel produced by mixing taxane dissolved in organic solvent, specifically ethanol, with polysorbate 80, other tensoactives and excipients like mannitol to further evaporate the solvent by spray drying. This formulation proposes a soluble solid having components in its formulation which can cause side effects such as those generated by polysorbate 80. Besides, it proposes heating the drug for its drying, a step which originates degradation products known by the art. On the other hand, the paclitaxel solution that can be produced following the teachings of said patent contains 80% of ethanol, which would make a lyophilization process impossible to be applied to generate the solid formulation proposed.

The application of patent US2004/0247624 A1 by Evan Charles Unger, et al. describes another method to formulate drugs poorly soluble in water. It proposes to elaborate a solid composition lyophilizing a filtrate solution of an organic solvent, the poorly soluble drug and at least a stabilizing agent which does not have a covalent bond with the drug. The only example with paclitaxel (example #3) describes a complex process of sonication and heating of a mixture of two polymers in high concentration, t-butanol and the drug until solubilization is reached. Subsequently lyophilization is proposed to obtain a powder. Finally, it describes redissolution of such powder with another tensoactive solution which finally cannot dissolve the whole solid as it states that there are visible particles. Moreover, heating the drug in the solution at 60° C. causes paclitaxel degradation. The application US2005/0152979 by Marc Besman et al. claims a lyophilized composition of a drug poorly soluble in water which contains said drug, a polymer and an agent to improve reconstitution. In spite of claiming paclitaxel and docetaxel as drugs suitable to be used by the invention, the tests it states are performed with a paclitaxel conjugate named CT-2103 which is an ester conjugate of alpha-poly-(L)glutamic acid and paclitaxel, of a much higher solubility in aqueous solutions than paclitaxel itself. Neither paclitaxel nor docetaxel dissolves in sodium phosphate aqueous solutions with the excipients and tensoactives described in this document.

The application of patent US2003/0099674 A1 by Andrew Chen describes a taxane composition solubilisable in water elaborated by the lyophilization of an emulsion of the drug in oil, also containing an anti-adhesion agent. An important degradation of taxanes in oils is observed in this patent, when they are subjected to a temperature of 60° C. for a month. This technology poses the problems described above as regards stability and macrophage reactions in the presence of emulsions injected in the body of mammals.

Other technologies like those used in taxane conjugates are described in documents such as the application of patent US2003/0147807 by Chun Li et al. which characterizes a taxane composition soluble in water. However, it refers to conjugates of paclitaxel and docetaxel attached to soluble polymers such as polyglutaminic acid, polyaspartic acid, etc.

Other patents evaluated as the state of the art related to the present invention were the following: US2005/0191323, WO9814174, U.S. Pat. No. 6,630,121, U.S. Pat. No. 6,607,784, WO2005/044225, US2006/0127420, US2006/0078619, US2004/0091541, US2003/0215496, US2001/0018072, U.S. Pat. No. 5,922,754 and WO2005025499.

The current state of the art offers a great number of solutions to obtain formulations of drugs of a low solubility in water. However, it was not possible to obtain in said solutions a solid composition of lyophilized taxane free from other compounds, mainly from those which modify its pharmacodynamics and chemical stability during storage.

To achieve the solubilization of these drugs of a poor solubility in aqueous media, a great number of methods have been proposed in the state of the prior art, such as intense agitation, heating, sonication, solvent evaporation, dialysis, spray-drying, emulsification/evaporation, micronisation, etc. The proposals involving lyophilization as part of the procedure which have already been described generally start from a mixture containing organic solvents, water, polymers, excipients, tensoactives, lipids, and lipoproteins among other components. These mixtures usually involve complex emulsification or dilution processes that often require heating, sonication, intense stirring and use of polymers specially designed, among other procedures.

The present invention provides a pharmaceutical formulation of two components, one constituted by a taxane solid composition, especially lyophilized docetaxel and paclitaxel, and the other component constituted by a liquid solubilizing composition.

Moreover, the present invention provides said solid composition of taxane, particularly docetaxel and paclitaxel, having extraordinary diluting features compared to pharmaceutical actives—either anhydrous or polyhydrated—available on the market. Said solid composition does not contain excipients, polymers or tensoactives, being free from any other component. This solid composition does not contain polyoxyethylated castor oil or polysorbate 80. Besides, it can be obtained by a simple procedure as it does not require heating, sonication or intense agitation, it is easy to repeat and it only involves two components: the pharmaceutical active and an organic solvent for lyophilization. The solid composition of the present invention is totally soluble in an aqueous tensoactive solution in less than a minute, free from the addition of an organic solvent.

The present invention provides an innovative method which allows lyophilization from a taxane solution in an organic solvent where said solution is obtained without the need of external, mechanic or thermal media to achieve a rapid and total dissolution. Said solution is subjected to lyophilization from which a lyophilized cake is obtained, containing only said taxane and traces of the lyophilization solvent. Thus, a pharmaceutical active of a great specific area and an extraordinarily enhanced solubility is obtained following a very simple procedure.

There are many taxane solubilization methods in the prior art, but in all cases the active pharmaceutical ingredient (API) is dissolved in an organic solvent, generally ethanol and subsequently other components are added namely tensoactives, water, excipients, etc. No literature has been found so as to infer that paclitaxel and docetaxel are solubilized in an aqueous solution in the absence of an organic solvent added to it.

Furthermore, it has been proved that the addition of organic solvents such as ethanol enhances the solubility of taxanes but affects their stability favoring anticipated precipitation of the drug when formulated in an aqueous solution of perfusion.

The present invention also provides a kit which allows the use of the formulation safely, since it decreases the risks of contaminating the drug and health-care staff who manipulates it, while it also facilitates the operation of drug administration. Said kit comprises the two compositions mentioned contained in sterile compartments, isolated from one another. Said kit also provides a syringe.

In a preferred version of the invention said syringe is prefilled and comprises said sterile compartments. Thus, the preparation of the perfusion formulation is simplified as both compartments come into contact, and by means of gentle movements, the dissolution of the lyophilized solid taxane composition is achieved in said solubilizing composition to be finally injected into the perfusion bag or flask containing saline solution or dextrose.

This prefilled syringe makes the operation of preparing the perfusion solution extremely easy and decreases execution time remarkably by eliminating to a considerable extent the risks of contamination either for the products or the operators and patients, which occurs with the product currently available.

The formulation of the invention enables the elimination of the habitual antihistamine pretreatment. This is due to the fact that this formulation is free from polysorbate 80 and polyoxyethylated castor oil. Furthermore, this formulation allows its administration in shorter times than the usual ones indicated in the practice, as long perfusion periods are necessary due to the presence of polysorbate 80. Thus, periods of less than 30 minutes would be necessary for the administration of taxanes using the formulation of the present invention.

Both, decreasing operative times for the preparation of the perfusion solution and, decreasing the perfusion times during the administration of the formulation of the invention provide the following benefits: a greater number of patients being treated in day care hospitals in the oncological clinical service of the current health system (either public or private), reduced operative costs on the basis of pharmacoeconomics, due to a better utilization of sanitary resources and prioritization of the safety of health-care providers involved in said manipulation (the risk of the stock solution which comes into contact with skin or mucosa of health-care providers is reduced to almost 0).

As a great number of adverse side effects is attributed to polysorbate 80, this new formulation can be administered to patients who cannot receive it today, namely patients with renal illnesses, cardiopathies, the elderly, and those with polysorbate 80 hypersensitivity, etc. It also can be administered to patients suffering from kidney cancer as it does not contain polysorbate 80 which causes fluid retention syndrome.

### SUMMARY OF THE INVENTION

The first object of the present invention is to solve the problems described in the prior state of the art and to provide a pharmaceutical formulation of taxane having an enhanced stability allowing storage at ambient temperature in tropical and subtropical climates, which facilitates the operations for the infusion preparation, is free from tensoactives and toxic excipients, particularly free from polyoxyethylated castor oil and polysorbate 80. Said formulation is intended for treatment in patients with hypersensitivity to said tensoactives, patients suffering from renal illnesses such as saline retention syndrome, the elderly and patients with cardiopathies, etc. This formulation comprises a solid composition of said lyophilized taxane and a solubilizing composition of said solid composition. In particular, it is directed to an injectable formulation of a taxane such as paclitaxel or docetaxel, suitable for use in parenteral infusion solutions in mammals, preferably humans.

A second object of the present invention is to provide said solid composition of a taxane, suitable to prepare pharmaceutical formulations of a great stability even at high temperatures (60° C.) as it is in solid state, essentially free from other components, of an enhanced solubility due to its high specific area and to its low apparent density, which can be solubilized by means of an aqueous solution of a tensoactive in the absence of an organic solvent. This solid composition comprises a lyophilizate from a solution of at least said taxane in an organic solvent.

A third object of the present invention is to provide said solubilizing composition of said lyophilized taxane solid composition suitable to be injected into parenteral infusion solutions comprising at least a polymeric tensoactive of a low toxicity.

A fourth object of the present invention is a procedure to prepare said lyophilized taxane solid composition comprising the following steps:

a) dissolving said taxane in lyophilization organic solvent

b) lyophilizing

c) optionally, drying

This procedure, in its preferred embodiment, also involves a sterilization step. Said sterilization step is preferably performed by means of a sterilizing filtration of the solution obtained in step a).

A fifth object of the present invention, a pharmaceutical perfusion solution, contains less than 1 mg/ml taxane in normal saline solution or dextrose solution and it also only contains Solutol®, essentially free from organic solvent, other tensoactives, oils, other polymers, solubility enhancers, preservatives and excipients.

A sixth object of the present invention is to provide a kit comprising: a first container holding said solid composition of taxane, a second container holding said solubilizing composition of said solid composition of taxane and a syringe.

A seventh object of the present invention is a prefilled syringe comprising said containers.

### BRIEF DESCRIPTION OF THE INVENTION

The pharmaceutical formulation of taxane, main object of this present invention, to be administered to mammals, mainly humans, preferably free from polysorbate 80 and free from polyethoxylated, comprises two compositions which are combined prior to its administration forming a transparent solution free from precipitates, where said compositions comprise: a solid composition of lyophilized taxane, preferably free from tensoactives, oils, polymers, solubility enhancers, preservatives and excipients; and a solubilizing composition of said solid composition of lyophilized taxane comprising at least one tensoactive. Said solid composition presents an apparent density lower than 0.1 g/ml, preferably from 0.004 g/ml and 0.05 g/ml, most preferably from 0.006 g/ml and 0.02 g/ml. Said solid composition is soluble in an aqueous solution of Solutol® HS 15 to 20% in less than a minute and in the absence of an added organic solvent. Furthermore, said solid composition is chemically stable at 60° C. for at least 28 days with a degradation of less than 1%. Moreover, said solid composition of the invention is obtainable by the lyophilization of a solution comprising a lyophilization organic solvent, selected from the group comprised by dioxane, acetic acid, dimethylsulphoxide or a mixture thereof, preferably dioxane or acetic acid, and a taxane at a concentration from 0.1 to 50% preferably 0.1 and 6%, preferably in the absence of tensoactives, oils, polymers, solubility enhancers, preservatives and excipients. Also, said solid composition has a residual concentration of lyophilization organic solvent lower than 8%, preferably less than 3%. Said taxane is selected from the group comprised by derivatives of baccatine III, 10-deacethilbaccatine III and the conjugates, salts, hydrates and solvates thereof, preferably docetaxel and paclitaxel. Said solubilizing composition comprises a polymeric tensoactive at a concentration from 1% to 100%, preferably from 5% to 40% and water in the absence of organic solvents. Said tensoactive is polymeric and selected from the set comprised by macrogol hydroxystearate such as Solutol®, poloxamer such as Lutrol® and polyvynilpyrrolidone or a mixture thereof. A preferred solubilizing composition of the invention comprises Solutol® HS 15 from 10% to 50% and water from 50% to 90% (P/P %). Said solubilizing composition dissolves said solid composition at a concentration of at least 4 mg/ml in the absence of precipitates for at least 2 hours.

The procedure for the preparation of said solid composition of taxane suitable to prepare pharmaceutical formulations for mammals, particularly humans, another object of the invention, comprises the following steps:

a) dissolving said taxane in organic solvent, in the absence of polymers, tensoactives, oils or excipients

b) sterilizing

c) lyophilizing

d) optionally, drying

A pharmaceutical perfusion solution, another object of the present invention contains at least 1 mg/ml of taxane in a normal saline solution or dextrose solution and, it contains only Solutol®, essentially free from organic solvents, other tensoactives, oils, other polymers, solubility enhancers, preservatives and excipients.

A kit of elements, a further object of the present invention, comprises a first container holding said lyophilized taxane solid composition, a second container holding said solubilizing composition of said taxane solid composition and a syringe. Preferably, said syringe is prefilled and comprises said first container and said second container.

A kit for the preparation of the injectable taxane formulation suitable to prepare parenteral infusion solutions for mammals, preferably humans, another object of the present invention, comprises a taxane lyophilized solid composition; a solubilizing composition of said solid composition of said taxane; a syringe that allows mixing said solubilizing composition with said solid composition of said taxane and obtaining a transparent and stable solution of taxane at a concentration of at least 4 mg/ml to be injected in a bag of parenteral infusion free from precipitation for at least 2 hours.

### DETAILED DESCRIPTION OF THE INVENTION

As a result of thorough research and a great number of laboratory tests, a new pharmaceutical formulation of drugs with poor solubility in water, particularly taxanes, has been developed. Said invention achieves the preservation of the active substances over long periods, maintaining its stability during its useful life with no need of being kept in cold chain for preservation, even in tropical or subtropical countries. This new formulation eliminates the common problems encountered today with some of the formulations of the state of the art concerning in situ precipitation and gelification occurring upon injection into the perfusion flasks or bags containing a saline or dextrose solution. In chemotherapy treatments by blood perfusion, this new formulation allows said taxane to be introduced into the body of mammals, especially humans, in the absence of components which modify its solubility or bioavailability, or form a complex or join the drug covalently. Moreover, the formulation of the invention is a sterile formulation by means of a sterilizing filtration procedure. This new pharmaceutical formulation of taxanes, which is the main object of the present invention, comprises both a solid composition of said taxane, lyophilized from a solution of organic solvents of lyophilization, as well as a solubilizing composition of said solid composition. An essential feature of this invention is that said solid composition of taxane is prepared by the lyophilization of a solution of said taxane in a lyophilization organic solvent. Said lyophilization organic solvents have a relatively high fusion point (above −10° C.) to allow solidification and further lyophilization, but lower than 25° C. to allow its work as a liquid at ambient temperature. Among others, acetic acid, dioxane and dimethylsulphoxide can be mentioned as appropriate lyophilization organic solvents which allow an easy and fast dissolution of taxane without having to heat, sonicate or agitate energetically. Taxanes are soluble in these lyophilization organic solvents in a wide range of concentrations of up to 50% p/p without precipitation during prolonged periods of time as a chemically and physically stable solution is produced. A mixture of these solvents can be used to realize the present invention.

Taxane solutions in such lyophilization organic solvents allow, in its elaboration process, a sterilizing filtration by means of 0.2μ filter. Said filtration is performed prior to lyophilization to yield a sterile powder as the solid composition of the invention. The solubilizing composition is also sterilized by filtration. Thus, solid sterilization is avoided, which involves complex and expensive procedures and poses risks to the drug.

Besides, these taxane solutions in organic solvents of lyophilization can be dosed in the liquid form in the container to be lyophilized, allowing to obtain from 5 to 200 mg per container, ready to be reconstituted before injection. The solid lyophilized composition resulting from these taxane solutions in organic solvent of lyophilization is a powder with a large specific surface area which enables a complete and very rapid dissolution in the solubilizing composition. Thus, a solid composition is obtained consisting in a liophylised cake or block of a good stability through time under high temperatures, above 25° C., remaining in a proper state without the need of cold chain. The stability tests performed, as shown in Example 6, proved that the solid composition of the invention withstands high temperatures, on the order of 60° C., for periods of at least a month with less than 1% degradation.

In addition, it has been proved that it is possible to use lyophilization excipients normally used in the art, such as mannitol, lactose, bile acids, gelatin, etc. The lyophilization excipient may be added to the taxane solution in lyophilization organic solvent, either in powder form or as an aqueous solution. These taxane solutions in lyophilization organic solvent withstand the addition of water up to a concentration of 30%.

Although it is possible to add water, lyophilization excipients or an acid (such as citric, lactic, tartaric, ascorbic, acetic, hydrochloric acid or a mixture thereof), neither addition is essential to achieve the object of the present invention. Therefore, in a preferred embodiment of the present invention, only the taxane solution is lyophilized in organic solvent of lyophilization, without any type of additives. This allows obtaining a solid composition of taxane of a large specific area, low apparent density and a taxane which is free from any interactions with other components.

Lyophilization in vials allows obtaining an adequate lyophilizate in organic solvent for lyophilization in concentrations from 6 mg/ml to 200 mg/ml.

The apparent density of the lyophilizate is defined as the quotient between the mass of the lyophilization cake in grams and its volume in milliliters. This variable has been carefully evaluated and after innumerable experiences the values of apparent density were obtained, in which the technical effects of the invention are possible, such as the easy reconstitution of said lyophilized taxane solid composition in an aqueous solution free from organic solvent. It has been proved that the solubility of the solid composition of the invention improves as its apparent density decreases. Therefore, the lower the apparent density of the lyophilization cake, the faster it dissolves. This effect can be observed in the results of Example 4. Likewise, if the apparent density is too low, the size of the container required is incompatible with sanitary manipulation.

These values of apparent density are lower than 0.1 gm/ml preferably between 0.004 gm/ml and 0.05 gm/ml, more preferably between 0.006 gm/ml and 0.02 gm/ml.

The content of residual solvent in the lyophilizate is normally lower than 8%, preferably below 3%.

In a lyophilization process, with secondary drying stages of 24 hours, the quantity amount of residual solvent is no greater than 8% for acetic acid and 3% for dioxane. These values can be reduced up to 3 and 1% increasing drying temperature up to 50° C. and extending the drying time for another 24/48 hours, without major problems of active drug degradation.

Laboratory tests have proved that the pharmaceutical active available in the market, particularly trihydrate or anhydrous docetaxel, cannot be dissolved directly neither in a tensoactive aqueous solution, nor in pure polysorbate 80, whereas the solid composition of the invention is easily soluble in an aqueous solution of Solutol® HS15 as well as in pure polysorbate 80. Also, it has been proved that said solid composition of the invention is rapidly soluble in a solution of polysorbate 80 (PS80): EtOH:water (25:9.75:65.25); unlike anhydrous docetaxel (API) available in the market.

An advantage of the solid composition of the invention is the remarkable improvement in the readiness to dissolve in solvents of the tensoactive and polymer group. Thus, said lyophilized solid composition can be easily dissolved in mixtures of Lutrol® F68, Lutrol® E400, Solutol® HS15, avoiding the use of polysorbate 80.

Among the objects of the present invention it can be stated the elimination of polysorbate 80, polyoxyethylated castor oil and organic solvents such as ethanol, present in the current market formulations of docetaxel and paclitaxel.

The solid composition of the invention, obtainable by lyophilization of taxanes in a solution of organic solvents of lyophilization, allows the solubilization of said taxanes rapidly not only in tensoactive aqueous solutions but also in pure tensoactives, such as polysorbate 80, in the absence of organic solvents added. It also allows the solubilization in aqueous solutions of polymers such as Solutol® HS15, Lutrol® F68, povidone, Lutrol® E400 and a mixture thereof. These formulations can be completely free from ethanol, mainly from polysorbates and polyoxyethylated castor oil.

In the state of the art, a usual way of accelerating the solubilization process of taxanes is the use of organic solvents such as ethanol. This is the alternative mostly used in the abundant literature addressing this technological field. In a first step, taxane is dissolved in the solvent and in a second stage the amphiphilic polymers, surfactant or tensoactives and the mixture thereof are added. The disadvantage of this alternative is the residual presence of solvent used and the lower stability of the solutions of the taxanes, especially docetaxel in the presence of ethanol. Besides, long periods of solubilization, sonication, intense agitation, temperature increase and other operations are needed to achieve a stable solution or dispersion. Nevertheless, the use of an organic solvent, which is not necessary for the present invention, can be utilized as an ingredient of said solubilizing composition.

The tensoactives suitable to be used in the present invention to formulate said solubilizing composition, among others, can be: polyetylglycol, polyvinylpirrolidone, poloxamer, hydroxypropylcellulose, polymethacritates, polysine, poly vynil alcohol, poly acrylic acid, ethylene polyoxide, hyaluronic acid, dextrane sulphate and its derivatives, calcium stearate, glyceron monostearate, cetoestearilic alcohol, emulsifying wax of cetomacrogol, sorbitan esters, alquilic eter of ethylene polyoxide, macrogol esters, as cetomacrogol 1000, derivates of ricine oil polyocyothylenic, polysorbates, polysorbate 80, fat acid esters of polyoxyethylenesorbitane (TWEEN), estearates of polyoxyothylene, sodic dodecilsulphate, calcium carboxymethylcellulose, sodium carboxymethylcellulose, methylcellulose phtalate of hydroxipropylmethylcellulose, non crystalline cellulose, Triton.

A preferred embodiment of the present invention comprises as tensoactives for said solubilizing composition Solutol® HS 15 (Polyethylene glycol 15-hydroxyestearate), a macrogol hydroxiestearate, Lutrol® F68, a poloxamer provided by the firm BASF, polyvynilpirrolidone, or mixtures thereof. In particular, Solutol® HS 15, Lutrol® F68 or the mixtures thereof are preferred.

Solutol® HS 15 is a hydroxystearate macrogol. It is also known by the following names: polyethylene glycol-15-hydroxystearate, polyethylene glycol 660-12-hydrostearate, macrogol-15-hydroxystearate, CAS No 70142-34-6 is a polymeric tensoactive used in injectables to solubilize hydrophobic actives and avoid sedimentation and recrystallization. Its low toxicity and extraordinary solubilizing power, allow its use in high concentrations. A very low histaminic release has been proved after the administration to mammals as compared to polysorbates (application of Solutol® HS 15—A Potent Solubiliser with a Low Toxicity’ F. Ruchatz). Some studies suggest that this solubilizer can present as desired side effect the reversion of multiple resistance of some carcinogenic cells as regards anticancer drugs. (K. H. Frömming et. al., Acta Pharm. Technol. 36(4). 1990, 214-220; J. S. Coon et. al., Cancer Res. 51 (3). 1991, 897-902; J. S. Coon, Proc. Am. Assoc. Cancer Res. 33. 1992, 484; D. Hoover et. al., Fundam. Appl. Toxicol. 14(1990), 589 pp.

The procedure for the preparation of the solid composition of taxane of the present invention comprises the following steps:

a) dissolving said taxane in organic solvent of lyophilization in the absence of polymers, tensoactives, oils or excipients.

b) lyophilizing

c) drying—optional

This procedure includes, in its preferred embodiment, a sterilization which is preferably performed by sterilizing filtration of the solution obtained in step a). Said sterilizing filtration is done with filtration membranes of materials which are inert to the organic solvents of lyophilization used in the present invention. Among the filtrating membranes suitable to be used are those of Teflon or nylon, being the preferred pore size 0.22 μm. Other sterilization methods such as gamma radiation, UV, etc. can be used.

Step a) of the procedure of the present invention is very simple, since it does not need energetic agitation, sonication, heating, addition of other solvents or another device used in the state of the art to achieve the solubilization of taxanes. Once the taxane solution in organic solvent of lyophilization is obtained, it is dosed in vials to lyophilize. This step is also simpler and faster than the ones already known in the state of the art as the solution in lyophilization organic solvent is much less viscose (1.2 cps) than the conventional polysorbate solutions (viscosity: around 400 cps). Therefore, not only production is simplified, but also production times are shortened applying the procedure of the present invention.

The variables involved in the operation of lyophilization, such as time, temperature, pressure, etc. can adopt a wide range of values.

Different modes to carry out this operation successfully are known in the state of the art. A reference is made of a possible particular manner to perform this operation, as it was done in the practice by the inventors, for descriptive purposes only:

After powder taxane is dissolved in the organic solvent of lyophilization, the solution is frozen in the container in which lyophilization is to be done; the frozen product is allowed to mature to improve its properties; lyophilization is performed at a tray temperature near the fusion point of the frozen product, which is generally reached at −20 to 20° C., a condensation temperature which allows sublimation of vapor into a solid, generally from −40° C. to −100° C. and a pressure in the condenser which is lower than the vapour pressure of the solvent in the lyophilization chamber.

Secondary drying is done at a tray temperature between 20 and 50° C., more especially between 25° C. and 35° C. and a total pressure lower than 1 mmHg, for not less than 8 hours.

In a preferred embodiment of the present invention, said formulation is prepared starting from a solid composition of lyophilized taxane, in a sterilized container and a solubilizing composition of Solutol® HS 15 in water, which after being mixed with one another, is injected into perfusion solution, for example normal saline solution or dextrose solution, to obtain a taxane solution stable for more than 2 hours, that is infused to patients undergoing oncological treatment.

The present invention also comprises a pharmaceutical perfusion solution containing less than 1 mg/ml of taxane in normal saline solution or dextrose solution and also contains only Solutol®, essentially free from organic solvents, other tensoactives, oils, other polymers, solubility enhancers, preservatives and excipients. This perfusion solution is the one prepared with the pharmaceutical formulation of taxane of the present invention. Essentially, free from organic solvents means that it does not have the addition of organic solvents such as ethanol to enhance solubility, and it can only contain small concentrations of lyophilization organic solvents which can remain from the process of preparation of the solid composition of the present invention. Besides, said pharmaceutical perfusion solution for the infusion of taxane in mammals, especially humans, for the treatment of cancer, is of a low toxicity, requiring shorter times for the perfusion process (less than 30 minutes) and does not require pretreatment with steroids or antihistamines since it is free from polysorbate 80, polyoxyethylated castor oil, emulsions or any other component.

Another preferred embodiment of the present invention comprises a kit consisting of a first container holding the solid composition of lyophilized taxane of the invention; a second container holding the solubilizing composition of the invention and a syringe.

In another preferred embodiment of the present invention said syringe is prefilled and contains said containers, which are independent from one another, with means for connecting said containers prior to the administration and, optionally, with a filter.

It is worth highlighting that other drugs which are acceptable for being used as active matter in the formulation of the present invention are: albuterol, adapalene, doxazosin mesylate, mometasone furoate, ursodiol, amphotericin, enalapril maleate, felodipine, nefazodone hydrochloride, valrubicin, albendazole, conjugated estrogens, medroxyprogesterone acetate, nicardipine hydrochloride, zolpidem tartrate, amlodipine besylate, ethinyl estradiol, omeprazole, rubitecan, amlodipine besylate/benazepril hydrochloride, etodolac, paroxetine hydrochloride, atovaquone, felodipine, podofilox, paricalcitol, betamethasone dipropionate, fentanyl, pramipexole dihydrochloride, Vitamin D.sub.3 and related analogues, finasteride, quetiapine fumarate, alprostadil candesartan, cilexetil, fluconazole, ritonavir, busulfan, carbamazepine, flumazenil, risperidone, carbemazepine, carbidopa/levodopa, ganciclovir, saquinavir, amprenavir, carboplatin, glyburide, sertraline hydrochloride, rofecoxib carvedilol, halobetasolproprionate, sildenafil citrate, celecoxib, chlorthalidone, imiquimod, simvastatin, citalopram, ciprofloxacin, irinotecan hydrochloride, sparfloxacin, efavirenz, cisapride monohydrate, lansoprazole, tamsulosin hydrochloride, mofafinil, azithromycin, clarithromycin, letrozole, terbinafine hydrochloride, rosiglitazone maleate, diclofenac sodium, lomefloxacin hydrochloride, tirofiban hydrochloride, telmisartan, diazapam, loratadine, toremifene citrate, thalidomide, dinoprostone, mefloquine hydrochloride, trandolapril, docetaxel, mitoxantrone hydrochloride, tretinoin, etodolac, triamcinolone acetate, estradiol, ursodiol, nelfinavir mesylate, indinavir, beclomethasone dipropionate, oxaprozin, flutamide, famotidine, nifedipine, prednisone, cefuroxime, lorazepam, digoxin, lovastatin, griseofulvin, naproxen, ibuprofen, isotretinoin, tamoxifen citrate, nimodipine, amiodarone, and alprazolam, amphotericin B, cyclosporine, etoposide, topotecan, melphalane, idarubicine, doxorubicin, vinorelbine, vinblastine, vinchristine.

Reference is made herein to concentrations weight/weight (w/w) when no explicit reference is made to any other type of magnitudes.

For a better understanding of the technical and functional aspects of the present invention, and without implying a restriction on the scope of this patent application, there follows a set of examples of application involving some of the alternatives comprised in the present invention and a set of comparative examples to assess the differences stated in the light of the prior technique.

### EXAMPLES

Materials: In the experiments glass vials type I, 20 mm-wide mouth, 22 mm body diameter, 27 mm of height, Nuova Ompi, with a nominal capacity of 7 ml were used. The 20 mm apyrogen caps for lyophilization were sterilized with butyl-bromide from Helvoet Pharma.

The solvents used were quality, Acetic acid, Merk, item 1.000632511; Dioxane TEDIA, item DR-0480; ethanol, Merck, item 1.009832511. Distilled water was water quality for injectables (WFI) according to USP and EP specifications. Solutol® HS 15 BASF Trade Mark, item No 51633963; Lutrol® F68 (Poloxamer 188) BASF, item No 51633115; Lutrol® E400 BASF, item No 51632267.

The lyophilization assays were carried out in a VIRTIS ADVANTAGE lyophilizer driven by a MENTOR unit. The determinations of HPLC were performed either in a HPLC BECKMAN System Gold with solvent module model 118, diodes detector Model 116 and autoinjector Wilson model 234, or in a HPLC Waters model 1525 with binary pumps and a diode array detector, model 2996, a forced circulation oven Alltech (column thermostat Jetstream 2 Plus) and automatic sampler Waters 717 Plus. The solvent content was analyzed by gaseous chromatography AGILENT TECHNOLOGIES 6890N GC system with injector AGILENT TECHNOLOGIES 7683 B series with a sampler PERKIN ELMER Head Space Turbo Matrix 40 and a Mass Selective Detector AGILENT TECHNOLOGIES 5973 Network Mass selective Detector.

### Example 1

Docetaxel in a quantity of 389 mg was dissolved in glacial acetic acid (previously added with 1% water and kept at 100° C. for 1 hour to hydrolyze all the acetic anhydrous present) to obtain 7.78 ml of solution (5% w/v). 0.20 ml were dosed (to obtain 10 mg of docetaxel per 7 ml flask; it was frozen at −18° C. for 10 hours, and lyophilized. A solid composition of docetaxel formed by a lyophilized powder in the form of a cake was obtained.

### Example 2

Preparation of docetaxel lyophilisates in acetic acid: anhydrous docetaxel solutions were prepared by direct dilution in acetic acid to obtain solutions of concentrations of 50 mg/ml, 40 mg/ml, 20 mg/ml, 13.3 mg/ml, 10 mg/ml. These solutions were dosed in vials to obtain individual doses of 20 mg of docetaxel in each case. Thus, 0.4 ml, 0.5 ml, 1 ml, 1.5 ml and 2 ml were dosed respectively of each concentration and were lyophilized to obtain a 20 mg/vial of docetaxel with a content of acetic acid lower than 3% and an apparent density of: 0.05, 0.04, 0.02, 0.013 and 0.01 respectively.

### Example 3

Preparation of docetaxel lyophilizates in dioxane: In the same way solutions of docetaxel were prepared in concentrations of 13.3 mg/ml and 10 mg/ml and were dosed 1.5 ml and 2 ml to obtain 20 mg of docetaxel in each vial with a dioxane content lower than 3% and an apparent density of 0.013 and 0.01 respectively.

### Example 4

Reconstitution or solubilization assays were performed with the solid lyophilized compositions obtained in Examples 2 and 3. The vials containing 20 mg of docetaxel each, obtained in said examples were added with a solubilizing composition formed by polysorbate 80:ethanol:water (25:9.75:65.25) and the time each test needed to completely solubilize the solid composition and result in a transparent liquid without precipitates was observed. The results are shown in the following table:

TABLE 1

| | | Volume of | |
|---|---|---|---|
| Lyophilizing | Apparent density of | solubilizing | Dissolution |
| organic solvent | the cake | composition | reconstitution time |
| Acetic acid | 0.050 | 2 ml | 12 min |
| Acetic acid | 0.020 | 2 ml | 2 min |
| Acetic acid | 0.013 | 2 ml | 1 min |
| Dioxane | 0.013 | 2 ml | 1 min |
| Dioxane | 0.010 | 2 ml | <1 min |

The same assay was performed with 20 mg of commercial (anhydrous) docetaxel and it was not possible to dilute it, not even with intense agitation, remaining thus in solid state.

### Example 5

Anhydrous docetaxel (1.02 g), purity 98.2%, was placed in a 250 ml container and dissolved in dioxane to reach a final volume of 100 ml. Once dissolved, 2 ml of this solution were dosed in 7 ml vials, which were pre-covered and lyophilized following a lyophilization cycle with freezing stages of −60° C. for 240 min, and lyophilization at −3° C. for 1500 min, at 100° C. for 1500 min and final drying at 30° C. for 2420 min. 48 samples of 20 mg docetaxel were obtained. Each sample appeared as a lyophilizate of a homogeneous aspect without adherence to the walls, had an apparent density of 0.01 g/ml and was easily soluble in the following solvent mixtures:

TABLE II

| | Volume of solubilizing | Dissolution |
|---|---|---|
| Solubilizing composition | composition | time |
| 1st-Solutol HS15:water | 2 ml | <1 min |
| (25%) | | |
| 2nd-PS80:EtOH:water | 2 ml | <1 min |
| (25:9.75:65.25) | | |
| 3rd-PS80:Lutrol E400:water | 2 ml | <2 min |
| (5:20:75) | | |
| 4th-PS80:Lutrol F68:EtOH:Water | 2 ml | <2 min |
| (12.5:12.5:10:65) | | |
| 5th-PS80:Solutol | 2 ml | <1 min |
| HS15:EtOH:Water | | |
| (12.5:12.5:10:65) | | |
| 6th solutol HS 15:Lutrol F68:water | 2 ml | <1 min |
| (10:2:88) | | |

In all these solubilizing compositions except the second, less polysorbate was used than in the formulation currently available. In the third, 5 times less quantity was used and in the fourth and fifth only half the quantity was used. The solubilizing composition of the 1^{st} and 6^{th} are the ones preferred in the present invention as they are free from polysorbate 80.

### Example 6

Stability tests on the solid composition of the invention were conducted at 60° C. using docetaxel as taxane, and a comparison was made with docetaxel in a solution of polysorbate 80 available on the market. The purity of docetaxel was measured using HPLC. Purity results of docetaxel measured as a percentage of the peak area are shown in the following table.

TABLE III

| | T = 0 | T = 3 | T = 7 | T = 14 | T = 21 | T = 28 |
|---|---|---|---|---|---|---|
| Product | days | days | days | days | days | days |
| Lyo docetaxel- | 99.24 | 99.21 | 99.08 | 98.94 | 98.68 | 98.37 |
| acetic. acid | | | | | | |
| Lyo docetaxel- | 99.54 | 99.35 | 99.04 | 98.27 | 98.65 | Not |
| Dioxane | | | | | | measured |
| Docetaxel in PS 80 | 98.8 | 64.8 | 63.6 | 48.2 | 47.3 | 38.7 |

Lyophilized docetaxel (“lyo docetaxel”) has a greater stability than the solutions of docetaxel in polysorbate 80 as it is used in the formulation currently available.
The solid composition of the present invention has a greater stability than docetaxel solutions in polysorbate 80 as they are used in the current formulation.

### Example 7

Docetaxel solid compositions obtained in Example 5 with different solubilizing compositions were reconstituted. After obtaining a transparent solution in less than a minute, 2 minutes were allowed for the foam to settle and the solution obtained was injected into a container holding a perfusion solution, either normal saline or dextrose.

TABLE IV

| | Perfusion | |
|---|---|---|
| Solubilizing solution | 0.475 mg/ml | Stability |
| 4 ml/Solutol HS15:Water | Dextrose/saline | At least 6 hours/at least 6 |
| (20:80) | | hours |
| 4 ml/Solutol HS15:Water | Dextrose/saline | At least 6 hours/at least 6 |
| (25:75) | | hours |
| 4 ml/Solutol HS15:Water | Dextrose/saline | At least 6 hours/at least 6 |
| (30:70) | | hours |

where each vial containing the solid composition of lyophilized docetaxel 20 mg was reconstituted with 4 ml of solubilizing composition.

### Example 8

Anhydrous docetaxel, purity 98.2%, in a quantity of 1.025 g was placed into a 250 ml container, and dissolved to a final volume of 80 ml of acetic acid. Once dissolved, it was dosed in 1.6 ml of this solution in vials of 7 ml. 48 doses of 20 mg each were obtained. Doses were pre-covered and taken to lyophilization following a cycle of freezing stages of −60° C. for 240 min, and lyophilization at −5° C. during 1500 min, at 5° C. during 1500 min and final drying at 30° C. during 2170 min. The lyophilizate resulted in a cake of a homogeneous form without adherence to the walls, with an apparent density of 0.0125 g/ml. The purity of docetaxel obtained by this process of lyophilization was of 99.2% measured as a percentage area, detected by UV at 232 nanometers, in HPLC using a stainless steel column Waters Simmetry C18, of 4.6 mm×15 cm, 5 microns and a mobile phase of acetonitrile:methanol:water (26:32:42, v:v:v) filtered and degassed.

### Example 9

Different vials of the solid composition of lyophilized docetaxel obtained in Example 8 were added by means of a needle 21G 1:1/2 solubilizing solution constituted by water for injection and different concentrations of Solutol® filtered by a membrane of 0.45μ. The result on the physical stability of the different experiences is shown in the following table, where the time spent by docetaxel to precipitate after mixing said solid composition and the solubilizing composition can be seen on the last column.

TABLE V

| | Solubilizing | | | |
|---|---|---|---|---|
| Docetaxel solid | composition | | Dissolution | Precipitation |
| composition | % Solutol ® | Volume | time | time |
| 1 vial (20 mg) | 20% | 2 ml | <1 min | 1 hour |
| 1 vial (20 mg) | 20% | 2 ml | <1 min | 2 hours |
| 1 vial (20 mg) | 20% | 2 ml | <1 min | 1 hour |
| 1 vial (20 mg) | 20% | 2 ml | <1 min | 2 hours |
| 1 vial (20 mg) | 20% | 2 ml | <1 min | 3 hours |
| 1 vial (20 mg) | 20% | 2 ml | <1 min | 8 hours |

### Example 10

Using a 21G 1:1/2 needle, different vials of lyophilized docetaxel obtained in Example 8 were added with different quantities of aqueous solutions of Solutol® filtered by 0.45 micron membranes. The solution obtained by stirring in less than a minute was allowed to settle for about 5 minutes to decrease the quantity of foam and it was injected into dextrose solution 5% and normal saline (0.9%) to obtain docetaxel solutions of 0.5 mg/ml ready for perfusion. The results on the physical stability of the different experiences are shown in the following table as ‘docetaxel precipitation time’

TABLE VI

| | Solubilizing | | Injection in | |
|---|---|---|---|---|
| Docetaxel solid | composition | | perfusion solution | Precipitation |
| composition | % Solutol | Volume | (0.5 mg/ml) | time |
| 1 vial (20 mg) | 20% | 2 ml | Dextrose | 2 hours |
| 1 vial (20 mg) | 20% | 2 ml | Saline | 2 hours |
| 1 vial (20 mg) | 20% | 4 ml | Dextrose | 6 hours |
| 1 vial (20 mg) | 20% | 4 ml | Saline | 6 hours |
| 1 vial (20 mg) | 20% | 2 ml | Dextrose | 3 hours |
| 1 vial (20 mg) | 20% | 2 ml | Saline | 3 hours |
| 1 vial (20 mg) | 20% | 4 ml | Dextrose | 8 hours |
| 1 vial (20 mg) | 20% | 4 ml | Saline | 8 hours |
| 1 vial (20 mg) | 20% | 2 ml | Dextrose | 3 hours |
| 1 vial (20 mg) | 20% | 2 ml | Saline | 3 hours |
| 1 vial (20 mg) | 20% | 4 ml | Dextrose | 8 hours |
| 1 vial (20 mg) | 20% | 4 ml | Saline | 8 hours |

### Example 11

A 5% paclitaxel solution in acetic acid was prepared (free from acetic anhydride). Vials of 5 ml were dosed with this solution; 10 mg of paclitaxel were obtained in each and they were subsequently frozen at −18° C. for 10 h. Vials were lyophilized with an oil vacuum pump at ambient temperature for 10 h and dried at 35° C. for 48 h, thus obtaining a paclitaxel solid composition as a cake.

1. A pharmaceutical formulation of taxane to be administered to mammals, particularly humans, comprising a combination of two compositions forming a transparent solution free from precipitates wherein said compositions comprise:
a) a solid composition of lyophilization taxane free from tensoactives and obtainable by lyophilization of a solution comprising a lyophilization organic solvent and a taxane; and
b) a solubilising composition of said solid composition of lyophilization taxane comprising at least one tensoactive.

2. The formulation of claim 1, wherein said solid composition is free from tensoactives, oils, polymers, solubility enhancers, preservatives, and excipients.

3. The formulation of claim 1, wherein said solid composition has a density lower than 0.1 g/ml.

4. The formulation of claim 1, wherein said solid composition has a density of 0.004 g/ml to 0.05 g/ml.

5. The formulation of claim 1, wherein said solid composition has a density of 0.006 g/ml to 0.02 g/ml.

6. The formulation of claim 1, wherein said solid composition is soluble in an aqueous solution of 20% Solutol® HS 15 in less than a minute, in the absence of an added organic solvent.

7. The formulation of claim 1, wherein said solid composition is chemically stable at 60° C. for at least 28 days with a degradation lower than 1%.

8. The formulation of claim 1, wherein said solid composition has a residual lyophilization organic solvent concentration lower than 8%.

9. The formulation of claim 1, wherein said solid composition has a residual lyophilization organic solvent concentration lower than 3%.

10. The formulation of claim 1, wherein said lyophilization organic solvent is selected from the group consisting of dioxane, acetic acid, dymethylsuphoxide, and a mixture thereof.

11. The formulation of claim 1, wherein the concentration of said taxane in said solution is from 0.1 to 50%.

12. The formulation of claim 1, wherein the concentration of said taxane in said solution is from 0.1 to 6%.

13. The formulation of claim 1, wherein said solution comprises only said lyophilization organic solvent and said taxane in the absence of tensoactives, oils, polymers, solubility enhancers, preservatives, and excipients.

14. The formulation of claim 1, wherein said lyophilization organic solvent comprises dioxane.

15. The formulation of claim 1, wherein said lyophilization organic solvent comprises acetic acid.

16. The formulation of claim 1, wherein said taxane is selected from the group consisting of baccatin III derivatives; 10-deacetilbaccatin III derivatives; conjugates, salts, hydrates and solvates of baccatin III derivatives; and conjugates, salts, hydrates and solvates of 10-deacetilbaccatin III derivatives.

17. The formulation of claim 1, wherein said taxane is selected from the group consisting of docetaxel and salts, hydrates, or solvates thereof.

18. The formulation of claim 1, wherein said taxane is selected from the group consisting of paclitaxel and salts, hydrates, or solvates thereof.

19. (canceled)

20. The formulation of claim 1, wherein said solubilizing composition comprises a polymeric tensoactive and water, in the absence of organic solvent.

21. The formulation of claim 1, wherein said solubilising composition comprises a tensoactive at a concentration of 1% to 100%.

22. The formulation of claim 1, wherein said solubilising composition comprises a tensoactive at a concentration of 5% to 40%.

23. The formulation of claim 20, wherein said polymeric tensoactive comprises a concentration of 1% to 100% and is selected from the group consisting of macrogol hydroxistearate, poloxamer, polyvinylpirrolidone, and a mixture thereof.

24. The formulation of claim 1, that is free from polysorbate 80.

25. The formulation of claim 1 that is free from polyoxyethylated castor oil.

26. The formulation of claim 20, wherein said polymeric tensoactive comprises Solutol® HS 15.

27. The formulation of claim 20, wherein said solubilising composition comprises Solutol HS 15 from 10 to 50% P/P % and water from 50% to 90% P/P %.

28. The formulation of claim 1, wherein said solubilising composition dissolves said solid composition at a concentration of at least 4 mg/ml in the absence of precipitates for at least 2 hours.

29. The formulation of claim 1, wherein the combination of two compositions, when injected into normal saline solution or dextrose solution for perfusion, is transparent in the absence of in situ gelification and stable in the absence of precipitation for at least two hours.

30. A solid composition of taxane, suitable for the preparation of pharmaceutical formulations for mammals, particularly humans, wherein said solid composition comprises lyophilization taxane free of tensoactives, oils, polymers, solubility enhancers, preservatives, and excipients.

31. The solid composition of claim 30, having a density of 0.001 g/ml to 0.1 g/ml.

32. The solid composition of claim 30, having a density of 0.004 g/ml to 0.05 g/ml.

33. The solid composition of claim 30, having a density of 0.0067 g/ml to 0.02 g/ml.

34. The solid composition of claim 30, wherein said solid composition is soluble in an aqueous solution of 20% Solutol® in less than a minute in the absence of added organic solvent.

35. The solid composition of claim 30, wherein said solid composition is chemically stable at 60° C. for at least 28 days with degradation lower than 1%.

36. The solid composition of claim 30, wherein said solid composition has a residual organic solvent concentration of lyophilization lower than 8%.

37. The solid composition of claim 30, wherein said solid composition has a residual lyophilization organic solvent concentration lower than 3%.

38. The solid composition of claim 30, wherein said solid composition is obtainable by the lyophilization of a solution comprising a taxane and an organic solvent of lyophilization selected from the group consisting of dioxane, acetic acid, diletysulphoxide, and a mixture thereof.

39. The solid composition of claim 30, wherein said solid composition is obtainable by the lyophilization of a solution comprising a taxane and an organic solvent of lyophilisation comprising diosane.

40. The solid composition of claim 30, wherein said solid composition is obtainable by the lyophilization of a solution comprising a taxane and an organic solvent of lyophilisation comprising acetic acid.

41. The solid composition of claim 30, wherein said solid composition is obtainable by the lyophilization of a solution comprising a taxane and an organic solvent of lyophilisation, said taxane being present in said solution at a concentration of 0.1% to 50%.

42. The solid composition of claim 30, wherein said solid composition is obtainable by the lyophilisation of a solution comprising a taxane and an organic solvent of lyophilisation, said taxane being present in said solution at a concentration of 0.1 to 6%.

43. (canceled)

44. The solid composition of claim 30, wherein said solid composition is obtainable by the lyophilisation of a solution comprising a taxane and an organic solvent of lyophilisation, said solution comprising only said organic solvent of lyophilisation and said taxane being in the absence of tensoactives, oils, polymers, solubility enhancers, preservatives, and excipients.

45. The solid composition of claim 30, wherein said taxane is selected from the group consisting of baccatin III derivatives; 10-deacetylbaccatin III derivatives; conjugates, salts, hydrates and solvates of baccatin III derivatives; and conjugates, salts, hydrates and solvates of 10-deacetylbaccatin III derivatives.

46. The solid composition of claim 30, wherein said taxane is selected from the group consisting of docetaxel and salts of docetaxel, hydrates of docetaxel and solvates thereof.

47. The solid composition of claim 30, wherein said taxane is selected from the group consisting of paclitaxel and salts of paclitaxel, hydrates of paclitaxel and solvates thereof.

48. A procedure for the preparation of a solid composition of taxane, suitable to prepare pharmaceutical formulations for mammals, mainly humans, comprising the following steps:
a) dissolving a taxane in a lyophilization organic solvent in the absence of tensoactives, oils, polymers, solubility enhancers, preservatives, and excipients;
b) lyophilising; and
c) optional drying.

49. The procedure of claim 48, further comprising a sterilization step.

50. The procedure of claim 48, further comprising a sterilization step, wherein said sterilization step comprises a sterilizing filtration of the solution obtained in step a).

51. The procedure of claim 48, wherein said taxane is selected from the group consisting of baccatin III derivatives; 10-deacetylbaccatin III derivatives; conjugates, salts, hydrates and solvates of baccatin III derivatives; and conjugates, salts, hydrates and solvates of 10-deacetylbaccatin III derivatives.

52. The procedure of claim 48, wherein said taxane is selected from the group consisting of docetaxel and salts of docetaxel, hydrates of docetaxel and solvates thereof.

53. The procedure of claim 48 wherein said taxane is selected from the group consisting of paclitaxel and salts of paclitaxel, hydrates of paclitaxel and solvates thereof.

54. A solubilising composition of solid compositions of taxanes suitable to prepare injectable pharmaceutical formulations for parenteral infusion into mammals, mainly humans, comprising at least one tensoactive free from organic solvent.

55. The solubilizing composition of claim 54, wherein said solubilizing composition comprises a polymeric non-ionic tensoactive and water in the absence of an organic solvent.

56. The solubilising composition of claim 54, wherein said solubilizing composition comprises a polymeric non-ionic tensoactive at a concentration of 0.1% to 50%.

57. The solubilizing composition of claim 54, wherein said solubilizing composition comprises a polymeric non-ionic tensoactive at a concentration from 10% to 40%.

58. The solubilizing composition of claim 54, wherein said tensoactive comprises Solutol® HS 15.

59. A pharmaceutical perfusion solution containing less than 1 mg/ml of taxane in saline solution or dextrose solution, and further comprising Solutol®, essentially free from organic solvent, other tensoactives, oils, other polymers, solubility enhancers, preservatives, and excipients.

60. The solution of claim 59, wherein said taxane comprises docetaxel.

61. A kit for the formulation of injectable taxane, comprising a first container containing a solid composition of lypolized taxane; a second container containing a solubilizing composition of said solid composition of lyophilized taxane; and a syringe.

62. The kit of claim 61, wherein said syringe is prefilled and comprises said first container and said second container.

63. A kit for the preparation of an injectable formulation of taxane, suitable to prepare parenteral infusion solutions for mammals, particularly humans, comprising a solid composition of a lyophilized taxane; a solubilizing composition of said solid composition; and a syringe for mixing said solubilizing composition with said solid composition to obtain a transparent and stable solution of taxane at a concentration of at least 4 mg/ml to be injected into the parenteral infusion bag free from precipitation for at least 2 hours.

64. The solid composition of claim 30, wherein said solid composition is obtainable by the lyophilisation of a solution comprising a taxane and an organic solvent of lyophilisation.
